(19) United States
(12) Patent Application Publication
(10) Pub. No.: US 2024/0401029 A1
(43) Pub. Date: Dec. 5, 2024

(54) PRE-LIBRARY DEPLETION OF NUCLEIC ACIDS

(71) Applicant: ARC BIO, LLC, Scotts Valley, CA (US)

(72) Inventor: Stephane B. GOURGUECHON, San Mateo, CA (US)

(21) Appl. No.: 18/734,997

(22) Filed: Jun. 5, 2024

Related U.S. Application Data
(60) Provisional application No. 63/506,270, filed on Jun. 5, 2023.

(51) Int. Cl. C12N 15/10

(52) U.S. Cl. CPC C12N 15/1068

(57) ABSTRACT

Provided herein are methods for enriching a sample for nucleic acid sequences of interest, methods of serially depleting a sample of nucleic acids, and related kits. In some embodiments, the methods comprise a pre-depletion comprising phosphatase treatment of nucleic acids, cleavage with a plurality of nucleic acid-guided nuclease-guide nucleic acid (gNA) complexes, and exonuclease digestion of the nucleic acids.

### CROSS-REFERENCE TO RELATED APPLICATIONS

This application claims priority to U.S. Provisional Application No. 63/506,270 filed on Jun. 5, 2023, the content of which is incorporated by reference in its entirety.

### BACKGROUND OF THE INVENTION

Many human clinical DNA samples, or sample libraries such as cDNA libraries derived from RNA, or extracted DNA samples taken from tissue, fluids, or other host material samples contain highly abundant sequences that have little informative value and increase the cost of sequencing. While methods have been developed to deplete these sequences (e.g., via hybridization capture), these methods are often time-consuming and can be inefficient. Moreover, hybridization capture often looks to capture the DNA sequences of interest while discarding the remaining sequences. As a result, depletion by hybridization capture is not a viable option when the DNA sequences of interest are not known in advance, e.g. when screening a sample to study all microbial or non-host DNA sequences.

While shotgun sequencing of human samples to study microbial DNA can be done, low levels of microbial DNA in many samples has precluded the shotgun sequencing of many complex and/or interesting samples, due to cost. This is true of, for example, a metagenomic analysis of a sample, where the sample contains more than one species of organism (eukaryotic, prokaryotic, or viral organisms). For example, DNA libraries derived from whole human blood often contain >99% human DNA. Therefore, to detect an infectious agent circulating in human blood from shotgun sequencing, one would need to sequence to very high coverage. Thus, much of the cost associated with sequencing human DNA samples for the non-human nucleic acids provides relatively little metagenomic data. As a result, many human tissue DNA samples are considered unsuitable for metagenomic sequencing merely because the data yield is low compared to the resources required. Thus, there is a need in the art to increase microbial DNA yield in high host DNA samples and specifically to increase the percent of microbial DNA being sequenced when sequencing high host endogenous (HHE) DNA samples.

Thus, there exists a need in the art to achieve a low-cost, efficient method and compositions for sequencing analysis of complex mixtures of nucleic acids.

### SUMMARY OF THE INVENTION

In one aspect of the current disclosure, methods of enriching a sample for sequences of interest are provided. The methods begin by collecting and preparing, providing or obtaining a sample comprising sequences of interest and targeted sequences for depletion. The sample is contacted with a phosphatase to remove 5′ phosphates on the sequences of interest and the targeted sequences for depletion and generate a sample comprising sequences lacking 5′ phosphates. The sample comprising sequences lacking 5′ phosphates is then contacted with a plurality of nucleic acid-guided nuclease-guide nucleic acid complexes, optionally, CRISPR/Cas system protein-gRNA complexes. The guide nucleic acids (gNAs) in the nucleic acid-guided nuclease-guide nucleic acid complexes, which are optionally gRNAs, are at least partially complementary (contain a targeting region complementary) to targeted sequences for depletion. In this step, the targeted sequences for depletion are cleaved by the nucleic acid-guided nuclease-guide nucleic acid complexes to generate a sample comprising sequences of interest and cleaved targeted sequences for depletion. The sample comprising the sequences of interest and cleaved targeted sequences for depletion is then contacted with at least one exonuclease capable of targeting 5′ phosphate containing sequences for degradation and this results in the cleaved targeted sequences for depletion being degraded by the exonuclease.

In the methods, the nucleic acids in the sample may be fragmented prior to being contacted with a phosphatase or after treatment with the exonuclease. In some embodiments, the enriched sample is treated with fragmentase or contacted with one or more CpG methylation-sensitive restriction enzymes to cut or nick sites lacking CpG methylation and generate a fragmented sample. After exonuclease treatment or fragmentation, adapters may be ligated onto to the resulting sequences, enriched in the sequences of interest to generate adapter-ligated sequences. The adapter-ligated sequences can then be amplified by using adapter-specific PCR, to further enrich the sample for the sequences of interest. In some embodiments, the one or more CpG methylation-sensitive restriction enzyme comprises at least one of AluI, DdeI, HpyCH4IV, RsaI, and Sau3AI. In some embodiments, the one or more CpG methylation-sensitive restriction enzyme comprises all five of AluI, DdeI, HpyCH4IV, RsaI, and Sau3AI. The methods may further comprise generating a double-stranded or single-stranded DNA library comprising the sequences of interest. The methods further comprise amplifying, sequencing, or cloning the DNA library.

In another aspect, methods of serially depleting nucleic acids are provided. The methods include taking the sample generated using the above-described method and repeating the steps of the method, including contacting with the phosphatase, contacting with the nucleic acid-guided nuclease-gNA complex and contacting with the exonuclease, two or more times. After the exonuclease treatment, the resulting samples may be fragmented by contact with one or more CpG methylation-sensitive restriction enzymes to generate cut sites or nick sites in the DNA at enzyme recognition sites that lack CpG methylation. Adapters may then be ligated to the cut sites or nick sites to generate adapter-ligated sequences. The one or more CpG methylation-sensitive restriction enzyme may include at least one of AluI, DdeI, HpyCH4IV, RsaI, and Sau3AI. In some embodiments, the one or more CpG methylation-sensitive restriction enzyme comprises all five of AluI, DdeI, HpyCH4IV, RsaI, and Sau3AI.

The samples may comprise host nucleic acid sequences targeted for depletion and non-host nucleic acid sequences of interest. The non-host nucleic acid sequences may comprise microbial nucleic acid sequences. The microbial nucleic acids sequences may be derived from bacteria, archaea, fungi, or a eukaryotic parasite. The gRNAs may have a target portion that is complementary to host nucleic acid sequences. The target sequences for depletion may be repetitive sequences. The methods may further comprise generating a double-stranded or single-stranded DNA library comprising the sequences of interest. The methods may further comprise amplifying, sequencing, or cloning the DNA library.

In another aspect, kits are provided. The kits include a phosphatase, a plurality of gNAs with a region complementary to targeted sequences for depletion and an exonuclease. The kits may further comprise one or more CpG methylation-sensitive restriction enzymes. The one or more CpG methylation-sensitive restriction enzyme may comprise at least one of AluI, DdeI, HpyCH4IV, RsaI, and Sau3AI. In some embodiments, all five of AluI, DdeI, HpyCH4IV, RsaI, and Sau3AI are included in the kits. The gNAs may comprise gRNAs with a region complementary to targeted sequences for depletion to allow these sequences to be targeted for cleavage using a nucleic acid-guided nuclease—gNA complex.

### BRIEF DESCRIPTION OF THE DRAWINGS

FIG. 1 shows a schematic for an exemplary pre-library depletion embodiment.

FIG. 2 shows a flow chart demonstrating steps of an embodiment of the disclosed methods and approximate time required to complete each step, in comparison to other depletion methods.

FIG. 3A and FIG. 3B show increased recovery of spiked-in microbial DNA using the disclosed pre-depletion methods compared to non-depleted controls. DRASH refers to methods comprising contacting nucleic acid mixtures with CpG methylation-sensitive restriction enzymes selected from the group consisting of AluI, DdeI, HpyCH4IV, RsaI, and Sau3AI (i.e., the DRASH enzymes). Ribo refers to cDNA encoding ribosomal RNA. 90K refers to one embodiment of the instant disclosure comprising 90,000 gNAs targeting human genomic DNA.

FIG. 4A and FIG. 4B show increased recovery of spiked-in microbial DNA using the disclosed pre-depletion methods compared to normal depletion. Microbes were spiked into normal plasma at a concentration of 1000 microbes/ml or 100 microbes/ml. Chromosome 15 is representative of total human genomic DNA.

FIG. 5 shows a comparison of negative control, pre-library depletion, and library depletion for low and high concentrations of an oligonucleotide (Oligo 298) and baculovirus, presented in reads per million (RPM) and genome equivalents per million (EvPM).

FIG. 6 shows fragment size analysis for pre-library depletion on a mock sample of lambda DNA and a 200 bp oligonucleotide, both untreated (F1) and subjected to second strand synthesis via random hexamers and klenow DNA polymerase (F2); mock samples were then treated with rSAP and cut with EcoRV (ER5) (F3 and F7), then treated with lambda exonuclease (LambExo) (F4 and F8), lambda exonuclease and exonuclease 1 (Exo1) (F5 and F9), or exonuclease 1 (F6 and F10).

### DETAILED DESCRIPTION OF THE INVENTION

Nucleotide sequencing technologies, in particular next generation sequencing technology, have provided researchers, physicians, and other biomedical professionals with an unparalleled ability to collect nucleotide sequence data from a wide variety of samples. However, in many cases samples, e.g., metagenomic samples, forensic samples, environmental samples, clinical samples, etc., sequencing data may not capture rare or difficult to sequence nucleic acids because of the sheer number of nucleic acids present in the sample. Moreover, to faithfully capture such sequences, the depth of sequencing, i.e., the number of reads per nucleotide sequence, may become cost prohibitive. Accordingly, the inventors have developed novel pre-depletion methods to enrich complex mixtures of nucleic acids for sequences of interest.

### Methods of Enriching a Sample for Sequences of Interest

In one aspect, the inventors disclose herein methods of enriching a sample for sequences of interest. The methods include providing a sample comprising sequences of interest and targeted sequences for depletion and contacting the sample with a phosphatase to remove 5′ phosphates on the sequences of interest and the targeted sequences for depletion to generate a sample comprising sequences lacking 5′ phosphates. The sample comprising sequences lacking 5′ phosphates is then contacted with a plurality of nucleic acid-guided nuclease (NAGN) guide nucleic acid (gNA) complexes, e.g., CRISPR/Cas system protein-gRNA complexes, wherein the gNAs include a portion complementary to targeted sequences for depletion. Through this step the targeted sequences are cleaved to generate a sample comprising sequences of interest and cleaved targeted sequences for depletion. The resulting sample from the cleaving step is then contacted with at least one exonuclease capable of targeting 5′ phosphate containing sequences for degradation and degrading the cleaved targeted sequences for depletion to generate a sample enriched or the sequences of interest.

Any sample comprising DNA may be used in the methods of the present invention. Suitable samples include, without limitation, biological samples, clinical samples, forensic samples, and environmental samples. Exemplary clinical and forensic samples include, but are not limited to, whole blood, plasma, serum, tears, saliva, mucous, cerebrospinal fluid, teeth, bone, fingernails, feces, urine, tissue and biopsy samples. In some embodiments, the DNA in the sample is fragmented prior to use in the method. The DNA for use in the method may be single or double stranded. A sample comprising ssDNA or RNA may be converted into dsDNA prior to use in the method. If RNA a reverse transcriptase reaction may be carried out to generate cDNA. The cDNA or other ssDNA in a sample may then be subjected to second strand synthesis by contacting the sample with random hexamers and Klenow DNA polymerase to generate a second complementary strand of the cDNA or ssDNA in the sample. This would allow for enrichment of viral sequences having either ssDNA or RNA genomes or the use of mRNA as a starting sample in the methods described herein. The DNA molecules in the sample may be about 20 to about 5000 base pairs (bp) in length, about 20 to about 1000 bp in length, about 20 to about 500 bp in length, about 20 to about 400 bp in length, about 20 to about 300 bp in length, about 20 to about 200 bp in length, about 20 to 100 bp in length, about 50 to about 5000 bp in length, about 50 to about 1000 bp in length, about 50 to about 500 bp in length, about 50 to about 400 bp in length, about 50 to about 300 bp in length, about 50 to about 200 bp in length, about 50 to 100 bp in length, about 100 to about 5000 bp in length, about 100 to about 1000 bp in length, about 100 to about 500 bp in length, about 100 to about 400 bp in length, about 100 to about 300 bp in length, or about 100 to about 200 bp in length.

In the disclosed methods, samples comprise “sequences of interest” and “targeted sequences for depletion”. Sequences of interest may be any sequence with any prevalence in the sample. However, in some embodiments, the sequences of interest comprise less than about 50%, less than about 40%, less than about 30%, less than about 20%, less than about 15%, less than about 10%, less than about 5%, less than about 1%, less than about 0.1%, less than about 0.001%, or less than about 0.0001% of the total nucleic acids in the sample.

The targeted sequences for depletion may be repetitive sequences or sequences with little informative value, e.g., mitochondrial DNA, mitochondrial RNA, ribosomal rRNA, ribosomal DNA, repetitive sequences, multi-copy sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, SINE repeats, LINE repeats, dinucleic acid repeats, trinucleic acid repeats, tetranucleic acid repeats, poly-A repeats, poly-T repeats, poly-C repeats, poly-G repeats, AT-rich sequences174774. or GC-rich sequences. In some embodiments, the targeted sequences for depletion comprise greater than about 99.999%, greater than about 99.99%, 99.9%, greater than about 90%, greater than about 80%, greater than about 70%, greater than about 60%, or greater than about 50%, about 99.999% to about 90%, about 99.999% to about 80%, about 99.999% to about 70%, about 99.99% to about 90%, about 99.99% to about 80%, about 99.9% to about 90%, about 99.9% to about 80%, about 99.9% to about 70% of the nucleic acids in the sample.

In the disclosed methods, sequences are contacted with a phosphatase to remove free phosphates, e.g., 5′ phosphates on DNA and RNA. In some embodiments, the phosphatase is shrimp alkaline phosphatase (SAP) or recombinant SAP (rSAP). rSAP is essentially identical to SAP, except that it is produced in a recombinant organism, e.g., E. coli or yeast, e.g., Pichia pastoris. Without being limited by any theory or mechanism, rSAP is an exemplary phosphatase for use in disclosed methods, in part, because of ease of use and because rSAP is heat sensitive, allowing the practitioner to inactivate the phosphatase before performing additional steps of the methods. Thus, the phosphatase treatment removes 5′ phosphates on all sequences present in the sample, i.e., sequences of interest and targeted sequences for depletion.

After removal of the 5′ phosphates, the samples comprising sequences lacking 5′ phosphates, are contacted with a plurality of nucleic acid-guided nuclease complexes, e.g., CRISPR/Cas system protein-gRNA complexes, which cleave sequences at targeted sites. The term “cleaving,” as used herein, refers to a reaction that breaks the phosphodiester bonds between two adjacent nucleotides in both strands of a double-stranded DNA molecule, thereby resulting in a double-stranded break in the DNA molecule. The term “cleavage site”, as used herein, refers to the site at which a double-stranded DNA molecule has been cleaved. Thus, the sequences that comprise cleavage sites are cleaved to become 2 or more sequences that comprise 5′ phosphates. By contrast, nucleic acids that do not comprise cleavage sites do not have 5′ phosphates after treatment with the nucleic acid-guided nuclease complexes.

Nucleic acid-guided nuclease-based enrichment methods are described in WO/2016/100955, WO/2017/031360, WO/2017/100343, WO/2017/147345, and WO/2018/227025, the contents of each of which are incorporated by reference herein in their entirety.

As used herein, a “nucleic acid-guided nuclease” is any nuclease that cleaves DNA, RNA or DNA/RNA hybrids, and that uses one or more guide nucleic acids (gNAs) to confer specificity. A nucleic acid-guided nuclease can be a DNA-guided DNA nuclease, a DNA-guided RNA nuclease, an RNA-guided DNA nuclease, or an RNA-guided RNA nuclease. A nucleic acid-guided nuclease can be an endonuclease or an exonuclease. A nucleic acid-guided nuclease may be naturally occurring or engineered. In some embodiments, the nucleic acid-guided nuclease is selected from the group consisting of Cas9, Cpf1, Cas3, Cas8a-c, Cas10, Cas13, Cas14, Cse1, Csy1, Csn2, Cas4, Csm2, Cm5, Csf1, C2c2, CasX, CasY, Cas14, and NgAgo. The nucleic acid-guided nuclease can be from any bacterial or archaeal species. For example, in some embodiments, the nucleic acid-guided nuclease is from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis Corynebacter diphtheria, Acidaminococcus, Lachnospiraceae bacterium, or Prevotella.

A “guide nucleic acid (gNA)” is a nucleic acid that targets a nucleic acid-guided nuclease to a specific genomic sequence via complementary base pairing. The gNAs used with the present invention comprise a sequence that is complementary to a portion of a DNA molecule that is targeted for depletion (i.e., the target sequence). The complementary portion of a gNA comprises at least 10 contiguous nucleotides, and often comprises 17-23 contiguous nucleotides that are complementary to the target sequence. The complementary portion of the gNA may be partially or wholly complementary to the target sequence. In some embodiments, the gNA is from 20 to 120 bases in length, or more. In certain embodiments, the gNA can be from 20 to 60 bases, 20 to 50 bases, 30 to 50 bases, or 39 to 46 bases in length. Various online tools and software environments can be used to design an appropriate gNA for a particular application. The gNA may comprise DNA and/or RNA. In some embodiments, the gNA is a chemically modified gNA. For example, the gNA may be chemically modified to decrease a cell's ability to degrade the gNA. Suitable chemically modified gNAs may include one or more of the following modifications: 2′-fluoro (2′-F), 2′-O-methyl (2′-OMe), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the gNA is composed of two molecules that base pair to form a functional gRNA: one comprising the region that binds to the nucleic acid-guided nuclease and one comprising a targeting sequence that binds to the target site. Alternatively, the gNA may be a single molecule comprising both of these components, e.g., a single guide RNA (sgRNA). In some embodiments, the gNAs comprise guide RNAs (gRNAs).

In the disclosed methods, after treatment with a plurality of nucleic acid-guided nuclease complexes, e.g., CRISPR/Cas 9-gRNA complexes, the sample comprises sequences of interest lacking 5′ phosphates and targeted sequences for deletion which were cleaved by the nucleic acid-guided nuclease and now comprise 5′ phosphates. The sample is then treated with an exonuclease. In some embodiments, the exonuclease is lambda exonuclease, which is available commercially, which removes nucleotides from (i.e., degrades) sequences comprising 5′ phosphates in a processive manner from 5′ to 3′. Thus, the targeted sequences for depletion comprising 5′ phosphates are degraded, thereby enriching the sample for sequences of interest.

The targeted sequences for depletion and the sequences of interest may be fragmented. In some embodiments, the targeted sequences for depletion and the sequences of interest are fragmented prior to contacting with the phosphatase in the method. In other embodiments, the targeted sequences for depletion and the sequences of interest are fragmented after the exonuclease treatment. The means of fragmenting may include use of physical shearing, treatment with a fragmentase or treatment with one or more CpG methylation-sensitive restriction enzymes. In some embodiments, the enriched sequences of interest are fragmented and the fragmented sample is used to create libraries for next generation sequencing or for other molecular biological techniques, e.g., plasmid library generation, qPCR, etc.

Following exonuclease treatment or fragmenting the resulting sample, in some embodiments, the method further comprises ligating adapters to the sequences of interest, thereby generating adapter-ligated sequences of interest. In some embodiments the adapters are linear. In some embodiments the adapters are circular. In various embodiments, the adapter may be a hairpin adapter i.e., one molecule that base pairs with itself to form a structure that has a double-stranded stem and a loop, where the 3′ and 5′ ends of the molecule ligate to the 5′ and 3′ ends of the double-stranded DNA molecule of the fragment, respectively. Alternately, the adapter may be a Y-shaped adapter ligated to one end or to both ends of a fragment, also called a universal adapter. Alternately, the adapter may itself be composed of two distinct oligonucleotide molecules that are base paired with one another. Additionally, a ligatable end of the adapter may be designed to be compatible with overhangs made by cleavage by a restriction enzyme, or it may have blunt ends or a 5′ T overhang. Such adapters may be useful in generating libraries or for preparing for high throughput sequencing.

The adapter may include double-stranded as well as single-stranded molecules. Thus, the adapter can be DNA or RNA, or a mixture of the two. Adapters containing RNA may be cleavable by RNase treatment or by alkaline hydrolysis. Adapters can be 10 to 100 bp in length although adapters outside of this range are usable without deviating from the present invention. In specific embodiments, the adapter is at least 10 bp, at least 15 bp, at least 20 bp, at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 85 bp, at least 90 bp, or at least 95 bp in length.

In certain cases, an adapter may comprise an oligonucleotide designed to match a nucleotide sequence of a particular region of the host genome, e.g., a chromosomal region whose sequence is deposited at NCBI's Genbank database or other databases. Such an oligonucleotide may be employed in an assay that uses a sample containing a test genome, where the test genome contains a binding site for the oligonucleotide. In further examples the fragmented nucleic acid sequences may be derived from one or more DNA sequencing libraries. An adapter may be configured for a next generation sequencing platform, for example for use on an Illumina sequencing platform or for use on an IonTorrents platform, or for use with Nanopore technology.

In other embodiments, the adapter sequence binds to a particular capture molecule enabling isolation of adapter-ligated DNA molecules. For example, an adapter can hybridize with a capture molecule comprising a complementary DNA sequence, or an adapter may include a tag (e.g., biotin) that binds to a particular capture molecule (e.g., streptavidin). Suitable tags include, without limitation, 6-Histidine (His), hemagglutinin (HA), cMyc, GST, Flag, V5, and NE tags.

In some embodiments, the adapter-ligated sequences of interest are amplified using adapter specific-PCR, thereby enriching the sample for the sequences of interest.

Combination of Pre-Depletion with Further Depletion Strategies

In some embodiments, the disclosed methods include further steps to deplete sequences targeted for depletion or to enrich target sequences. As noted above, the samples after the exonuclease treatment may be contacted with one or more CpG methylation-sensitive restriction enzymes to generate cut sites or nick sites in the sequences at enzyme recognition sites that lack CpG methylation. After the contacting step, adapters can be ligated on to the cut sites or nick sites to generate adapter-ligated sequences. This step further enriches sequences that lack methylation such as microbial sequences.

“CpG methylation” is DNA methylation that occurs at a CpG site. A “CpG site” is a region of DNA wherein a cytosine nucleotide is followed by a guanine nucleotide in the 5′→3′ direction, separated by one phosphate group (which link nucleosides together to form DNA). In CpG methylation, the cytosine in the CpG dinucleotide is methylated to form 5-methylcytosine via addition of a methyl group by a DNA methyltransferase. CpG methylation plays a critical role in regulating gene expression. For example, the presence of multiple methylated CpG sites within promoters causes stable silencing of genes. In cancers, loss of gene expression occurs about 10 times more frequently by hypermethylation of promoters than by DNA mutations. Thus, CpG methylation status can be used as an indicator of gene activity or to distinguish between diseased and healthy states.

The DNA of mammals contains substantially higher levels of CpG methylation than the DNA of microorganisms, e.g., pathogens. Thus, by enriching for or depleting a sample of CpG methylated DNA, the present methods can be used to, for example, distinguish between mammalian DNA and the DNA of a pathogenic organism. For example, the methods can be used to enrich for the DNA of a pathogenic organism that is present within the mammalian host. Thus, in some embodiments, the sample comprises host nucleic acids targeted for depletion and non-host nucleic acid sequences of interest. The non-host nucleic acid sequences of interest may comprise microbial sequences of interest. The microbial nucleic acid sequences of interest may be derived from bacteria, archaea, fungi, or a eukaryotic parasite. The sample comprises DNA from a mammalian organism (host) and DNA from a pathogenic organism (non-host). Suitable mammalian organisms include, without limitation, humans, horses, sheep, cows, pigs, donkeys, cats, dogs, gerbils, mice, rats, and monkeys. In some embodiments, the mammalian organism is a human. Suitable pathogenic organisms include bacteria, yeast, viruses, and parasites. The methods of the present disclosure can be used to distinguish between human host DNA and commensal or mutualistic microorganism's DNA. Thus, in some embodiments, the sample comprises DNA from a mammalian organism and DNA from a commensal or mutualistic microorganism.

In the disclosed methods, samples are contacted with one or more CpG methylation-sensitive restriction enzymes to generate cut sites or nick sites in the DNA at enzyme recognition sites. As used herein, the term “CpG methylation-sensitive restriction enzyme” refers to a restriction enzyme that is “sensitive” to the presence of CpG methylation within its cognate recognition site or adjacent to its cognate recognition site (e.g., within 1-50 nucleotides), wherein “sensitive” means that the activity of the enzyme is altered by the presence of CpG methylation in the recognition site. The term “recognition site”, as used herein, refers to a specific DNA sequence that is recognized by a restriction enzyme. Some restriction enzymes cut within their recognition sites, while others cut adjacent to their recognition sites (e.g., within 1-105 nucleotides of the recognition site). In some embodiments, the recognition site is between 3-20 bp in length. However, in preferred embodiments, the recognition site is relatively short (e.g., 3-5 bp in length), such that the CpG methylation-sensitive restriction enzyme cleaves the DNA with greater frequency.

In the present methods, the CpG methylation-sensitive restriction enzyme(s) are used to generate cuts or nicks at their cognate recognition sites. As used herein, the term “cutting” refers to a reaction that breaks the phosphodiester bonds between two adjacent nucleotides in both strands of a double-stranded DNA molecule, resulting in a double-stranded break. And the term “cut site” refers to a site at which a DNA molecule has been cut. In contrast, the term “nicking” refers to a reaction that breaks the phosphodiester bond between two adjacent nucleotides in only one strand of a double-stranded DNA molecule, resulting in a single-stranded break. And the term “nick site” refers to a site at which a DNA molecule has been nicked.

The one or more CpG methylation-sensitive restriction enzymes may comprise a mixture of at least 2, at least 3, at least 4, at least 5, at least 6, at least 7, at least 8, at least 9, or at least 10 different CpG methylation-sensitive restriction enzymes. The activity of the one or more CpG methylation-sensitive restriction enzymes is blocked by CpG methylation within or adjacent to its cognate recognition site. Such enzymes cleave DNA at recognition sites that lack CpG methylation, and do not cleave or cleave at reduced levels at recognition sites that contain CpG methylation. Suitable CpG methylation-sensitive restriction enzymes that cannot cleave at genomic sites that are CpG methylated include, without limitation, AatII, AccII, AluI, Aor13HI, Aor51HI, BspT104I, BssHII, Cfr10I, ClaI, CpoI, DdeI, Eco52I, HaeII, HapII, HhaI, HpyCH4IV, MiuI, NaeI, NotI, NruI, NsbI, Nt.CviPII, PmaCI, Psp1406I, PvuI, RsaI, SacII, SalI, SmaI, SnaBI, and Sau3AI.

The inventors have previously developed a mixture of five CpG methylation-sensitive restriction enzymes that are blocked by CpG methylation, i.e., DdeI, RsaI, AluI, Sau3AI, and HpyCH4IV, which are referred to collectively herein as “DRASH enzymes”. Thus, in some embodiments, the one or more CpG methylation-sensitive restriction enzyme comprises at least one of the five DRASH enzymes. Because each of the DRASH enzymes has a different recognition sequence at which it cleaves unmethylated DNA, the use of multiple DRASH enzymes results in greater genomic coverage. Thus, in some embodiments, the one or more CpG methylation-sensitive restriction enzymes comprise at least two, at least three, at least four, or all five of the DRASH enzymes.

In some embodiments, the disclosed methods further comprise after step (d) contacting the sample with an exonuclease specific for single-stranded DNA. In some embodiments, the exonuclease specific for single-stranded DNA is exonuclease I. Thus, in some embodiments, the targeted sequences for depletion are further degraded by the additional treatment with an exonuclease specific for single-stranded DNA.

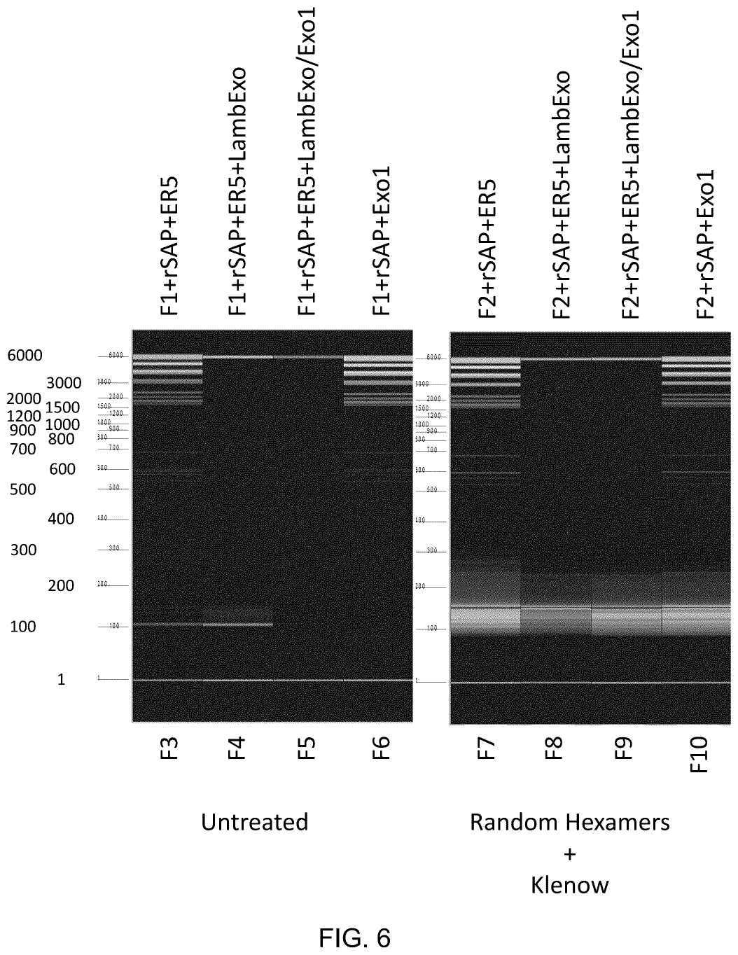

In some embodiments, the sequences of interest comprise single-stranded DNA. Therefore, to protect the single-stranded DNA sequences of interest from digestion by a single-stranded DNA-specific exonuclease, e.g., exonuclease I, second-strand DNA synthesis is performed on the sample prior to exonuclease I digestion (FIG. 6), suitably prior to treatment with the phosphatase. Briefly, the sample is contacted with Klenow polymerase, which retains 3′-5′ exonuclease activity, but is deficient in 5′-3′ exonuclease activity, and random primers to initiate second-strand synthesis, thereby generating double stranded DNA from the single-stranded DNA sequences of interest and protecting said sequences from digestion by exonuclease I. Prior to treatment with the exonuclease to remove single stranded sequences the sample may be treated with a blunt-end generating restriction enzyme.

The disclosed methods are advantageous for the enrichment of sequences of interest from a wide variety of sources. Therefore, in some embodiments, the sample is selected from the group consisting of a human sample, clinical sample, forensic sample, an environmental sample, a metagenomic sample, and a food sample. In some embodiments, the sample is selected from whole blood, plasma, serum, tears, saliva, mucous, cerebrospinal fluid, teeth, bone, fingernails, feces, urine, tissue, and a biopsy.

### Methods of Serially Depleting Nucleic Acids

In another aspect of the current disclosure, methods of serially depleting nucleic acids are provided. The methods the pre-depletion method above but allows for more than a single round of depletion. A sample comprising sequences of interest and targeted sequences for depletion is contacted with a phosphatase to generate a sample comprising sequences lacking 5′ phosphates. The sample lacking 5′ phosphates is then contacted with a plurality of nucleic acid-guided nuclease-gNA (CRISPR/Cas system protein-gRNA complexes), wherein the gNAs include a portion complementary to targeted sequences for depletion, and whereby the targeted sequences are cleaved to generate a sample comprising sequences of interest and cleaved targeted sequences for depletion The resulting sample is then contacted with at least one exonuclease capable of targeting 5′ phosphate containing sequences for degradation and degrading the cleaved targeted sequences for depletion. The resulting samples are then fragmented. In one embodiment, fragmenting is with one or more CpG methylation-sensitive restriction enzymes to generate cut sites or nick sites in the DNA at enzyme recognition sites that lack CpG methylation. The fragmented sample is then used in the methods again by treating with phosphatase, cleaving with the NAGN-gNA followed by exonuclease treatment to further enrich the sequences of interest. These steps can be repeated multiple times, such as 1-10 times, 1-5 times, 2-4 times or any combination thereof, e.g., 1 time, 2 times, 3 times, 4 times, 5 times, 6 times, 7 times, 8 times, 9 times, 10 times, or more. Once the depletion is complete and the sequences of interest are enriched then adapters can be ligated to the cut sites or nick sites to generate adapter-ligated sequences of interest. In some embodiments, the phosphatase is shrimp alkaline phosphatase (SAP) or recombinant SAP (rSAP). In some embodiments, degrading the cleaved targeted sequences comprises contacting the sample with an exonuclease. In some embodiments, the exonuclease is lambda exonuclease.

In some embodiments, the sequences of interest comprise less than 50% of the nucleic acids in the sample, less than 40% of the sample, less than 30% of the sample, less than 20% of the sample, less than 10% of the sample, less than 5% of the sample, less than 2% or the sample or even less than 1% of the sample, e.g., less than 0.1% of the sample, less than 0.01% of the sample, less than 0.001% of the sample, less than 0.0001% of the sample, less than 0.00001% of the sample, or less than 0.000001% of the sample.

In some embodiments, the one or more CpG methylation-sensitive restriction enzyme comprises a restriction enzyme selected from the group consisting of AatII, AccII, AluI, Aor13HI, Aor51HI, BspT104I, BssHII, Cfr10I, ClaI, CpoI, DdeI, Eco52I, HaeII, HapII, HhaI, HpyCH4IV, MluI, NaeI, NotI, NruI, NsbI, Nt.CviPII, PmaCI, Psp1406I, PvuI, RsaI, SacII, SalI, SmaI, SnaBI, and Sau3AI. In some embodiments, the one or more CpG methylation-sensitive restriction enzyme comprises at least one of AluI, DdeI, HpyCH4IV, RsaI, and Sau3AI. In some embodiments, the one or more CpG methylation-sensitive restriction enzyme comprises or consists of all five of AluI, DdeI, HpyCH4IV, RsaI, and Sau3AI.

In some embodiments, the sample is selected from the group consisting of a human sample, clinical sample, a forensic sample, an environmental sample, a metagenomic sample, and a food sample. In some embodiments, the sample comprises host nucleic acid sequences targeted for depletion and non-host nucleic acid sequences of interest. In some embodiments, the non-host nucleic acid sequences comprise microbial nucleic acid sequences. In some embodiments, the microbial nucleic acids sequences are derived from bacteria, archaea, fungi, or a eukaryotic parasite.

In some embodiments, the gRNAs are complementary to DNA corresponding to ribosomal RNA sequences, sequences encoding mitochondrial DNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

In some embodiments, the sample is selected from whole blood, plasma, serum, tears, saliva, mucous, cerebrospinal fluid, teeth, bone, fingernails, feces, urine, tissue, and a biopsy.

In some embodiments, the method further comprises generating a double-stranded or single-stranded DNA library comprising the sequences of interest. In some embodiments, the method further comprises amplifying, sequencing, or cloning the DNA library.

### Kits

In another aspect of the current disclosure, kits are provided. In some embodiments, the kits comprise a. a phosphatase, b. a plurality of gNAs complementary to targeted sequences for depletion, and c. an exonuclease.

In some embodiments, the kits further comprise: d. one or more CpG methylation sensitive restriction enzymes selected from the group consisting of AatII, AccII, AluI, Aor13HI, Aor51HI, BspT104I, BssHII, Cfr10I, ClaI, CpoI, DdeI, Eco52I, HaeII, HapII, HhaI, HpyCH4IV, MiuI, NaeI, NotI, NruI, NsbI, Nt.CviPII, PmaCI, Psp1406I, PvuI, RsaI, SacII, SalI, SmaI, SnaBI, and Sau3AI. In some embodiments, the one or more CpG methylation-sensitive restriction enzyme comprises at least one of AluI, DdeI, HpyCH4IV, RsaI, and Sau3AI. In some embodiments, the one or more CpG methylation-sensitive restriction enzyme comprises or consists of all five of AluI, DdeI, HpyCH4IV, RsaI, and Sau3AI. In some embodiments, the phosphatase is shrimp alkaline phosphatase (SAP) or recombinant SAP (rSAP). In some embodiments, the exonuclease is lambda exonuclease or exonuclease 1 (Exo1).

In some embodiments, the gNAs comprise gRNAs. In some embodiments, the targeted sequences for depletion comprises human DNA sequences. n some embodiments, the targeted sequences for depletion comprise ribosomal RNA sequences, mitochondrial RNA or DNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

### Definitions

The term “sample” as used herein relates to a material or mixture of materials, typically, although not necessarily, in liquid form, containing one or more analytes of interest.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses, or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “nucleic acids” and “polynucleotides” are used interchangeably herein. Polynucleotide is used to describe a nucleic acid polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally occurring nucleotides include guanine, cytosine, adenine and thymine (G, C, A and T, respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability but retain an ability to base pair with naturally occurring C and G residues, respectively. Unstructured nucleic acid is described in US20050233340, which is incorporated by reference herein for disclosure of UNA.

The term “hybridization” refers to the process by which a strand of nucleic acid joins with a complementary strand through base pairing as known in the art. A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

The term “duplex,” or “duplexed,” as used herein, describes two complementary polynucleotides that are base-paired, i.e., hybridized together.

The term “amplifying” as used herein refers to generating one or more copies of a target nucleic acid, using the target nucleic acid as a template.

The term “depleting,” with respect to a genome, refers to the removal of one part of the genome from the remainder of the genome to produce a product that is isolated from the remainder of the genome. The term “depleting” also encompasses removal of DNA from one species while retaining DNA from another species.

The term “genomic region,” as used herein, refers to a region of a genome, e.g., an animal or plant genome such as the genome of a human, monkey, rat, fish or insect or plant. In certain cases, an oligonucleotide used in the method described herein may be designed using a reference genomic region, i.e., a genomic region of known nucleotide sequence, e.g., a chromosomal region whose sequence is deposited at NCBI's Genbank database or other databases, for example.

The term “genomic sequence,” as used herein, refers to a sequence that occurs in a genome. Because RNAs are transcribed from a genome, this term encompasses sequence that exist in the nuclear genome of an organism, as well as sequences that are present in a cDNA copy of an RNA (e.g., an mRNA) transcribed from such a genome.

The term “ligating,” as used herein, refers to the enzymatically catalyzed joining of the terminal nucleotide at the 5′ end of a first DNA molecule to the terminal nucleotide at the 3′ end of a second DNA molecule.

If two nucleic acids are “complementary,” each base of one of the nucleic acids base pairs with corresponding nucleotides in the other nucleic acid. The term “complementary” and “perfectly complementary” are used synonymously herein.

The term “sequencing,” as used herein, refers to a method by which the identity of consecutive nucleotides of a polynucleotide are obtained.

The term “next-generation sequencing” refers to the so-called parallelized sequencing-by-synthesis or sequencing-by-ligation platforms, for example, those currently employed by Illumina, Life Technologies, and Roche, etc. Next-generation sequencing methods may also include nanopore sequencing methods or electronic-detection based methods such as Ion Torrent technology commercialized by Life Technologies.

The term “metagenomics sample” refers to a sample that contains more than one species of organism (eukaryotic, prokaryotic, or viral organisms).

The term “metagenomics analysis” refers to the analysis of a metagenomics sample.

### Guide Nucleic Acids

Provided herein are guide nucleic acids (gNAs), wherein the gNAs include a portion complementary to (selective for, can hybridize with) targeted sites or targeted sequences in the nucleic acids, for example in genomic DNA from a host. In one embodiment, the present invention provides a guide RNA library which comprises a collection of gRNAs, configured to hybridize with a nucleic acid sequence targeted for depletion or partitioning.

In one embodiment, the gRNAs are selective for target nucleic acids (or targeted sequences) in a sample but are not selective for sequences of interest in the sample.

In one embodiment, the gRNAs are selective for host nucleic acids in a biological sample from a host but are not selective for non-host nucleic acids in the sample from a host. In one embodiment, the gRNAs are selective for non-host nucleic acids from a biological sample from a host but are not selective for the host nucleic acids in the sample. In one embodiment, the gRNAs are selective for both host nucleic acids and a subset of the non-host nucleic acids in a biological sample from a host. For example, where a complex biological sample comprises host nucleic acids and nucleic acids from more than one non-host organisms, the gRNAs may be selective for more than one of the non-host species. In such embodiments, the gRNAs are used to serially deplete or partition the sequences that are not of interest. For example, saliva from a human contains human DNA, as well as the DNA of more than one bacterial species, but may also contain the genomic material of an unknown pathogenic organism. In such an embodiment, gRNAs directed at the human DNA and the known bacteria can be used to serially deplete the human DNA, and the DNA of the known bacteria, thus resulting in a sample comprising the genomic material of the unknown pathogenic organism.

In an exemplary embodiment, the gRNAs are selective for human host DNA obtained from a biological sample from the host, but do not hybridize with DNA from an unknown organism (e.g. pathogen(s)) also in the sample.

In some embodiments, the gRNAs are selective for a target nucleic acid sequences which are followed by Protospacer Adjacent Motif (PAM) sequences that can be bound by a Cas9. In some embodiments, the sequence of the gRNAs is determined by the CRISPR/Cas system protein type. For example, in various embodiments the gRNAs may be tailored to different Cas9 types as the PAM sequence can vary by the species of the bacteria from which Cas9 is derived. In some embodiments, more than one Cas system protein and gNA combination is used to allow for differential or multiple targeting and removal of additional targeted sequences for depletion. gRNAs can range in size for example, from 50-250 base pairs. For example, a gRNA can be at least 50 bp, 55 bp, 60 bp, 65 bp, 70 bp, 75 bp, 80 bp, 85 bp, 90 bp, 95 bp, 100 bp, 110 bp, 120 bp, 125 bp, 130 bp, 140 bp, 150 bp, 160 bp, 170 bp, 175 bp, 180 bp, 190 bp, or 195 bp. In specific embodiments, the gRNA is 80 bp, 90 bp, 100 bp, or 110 bp. Each target-specific gRNA comprises a base pair sequence that is complementary to a pre-defined site in a target nucleic acid that is followed by a Protospacer Adjacent Motif or (PAM) sequence that can be bound by a CRISPR/Cas system protein, for example a Cas9 protein, derived from a bacterial species. In specific embodiments, the base pair sequence of the gRNA that is complementary to a pre-defined site in a target nucleic acid is 15, 16, 17, 18, 19, 20, 25, 30, 35, 40, 45, or 50 base pairs.

The present invention also provides for gRNA libraries. A gRNA library can comprise a number of different species-specific gRNAs each, configured to hybridize with (be selective for) a nucleic acid sequence being targeted for depletion or partitioning. Each gRNA includes a target-specific guide sequence and a stem loop binding site that is formed to bind with a particular CRISPR/Cas system protein, for example with a Cas9 protein. The library can comprise a plurality of different guide RNAs, each having a different 15 to 20 base pair sequence that is complementary to a different pre-defined site in the nucleic acid being targeted, that is followed by an appropriate PAM sequence that can be bound by a CRISPR/Cas system protein. For each guide RNA the PAM sequence is present in the pre-defined DNA target sequence of the nucleic acid of interest but is not present in the corresponding target specific guide sequence.

Generally according to the present invention, any nucleic acid sequence in a genome of interest, with a pre-defined target sequence followed by the appropriate PAM sequence can be hybridized by a corresponding guide RNA provided in the guide RNA library and bound by CRISPR/Cas system protein, for example Cas9. In various embodiments the gRNA library may be tailored to different CRISPR/Cas system proteins, for example different Cas9 types since the PAM sequence can vary by the species of the bacteria from which protein is derived.

Without being limited to theory, the distance between gRNAs to arrive at >95% cleavage of the target nucleic acid can be computed, if the gRNAs display ˜100% efficacy: this can be computed by measuring the distribution of library size and determining the mean, N and the standard deviation SD; N-2SD=minimum size for >95% of the library, ensuring that there is one guide RNA per fragment of this size to ensure >95% depletion. This can also be described as the Maximum distance between guide RNAs=Mean of library size—2× (standard deviation of library size).

In the embodiments provided herein a gRNA library can be amplified to include a large number of copies of each different gRNAs as well as a large number of different gRNAs as may be suitable for the desired depletion results. The number of unique gRNAs in a given guide RNA library may range from 1 unique gRNAs to as many as 10¹⁰ unique gRNAs or approximately 1 unique guide RNA sequence for every 2 base pairs in the human genome. In some embodiments, the library comprises, at least 10² unique gRNAs. In some embodiments, library comprises at least 10², at least 10³, at least 10⁴, at least 10⁵, at least 10⁶, at least 10⁷, at least 10⁸, at least 10⁹, at least 10¹⁰ unique gRNAs. In one exemplary embodiment, the library comprises about 10⁵ unique gRNAs.

In the embodiments provided herein, the methods comprise contacting a sample comprising nucleic acids with a plurality of CRISPR/Cas9 system protein-gRNA complexes, wherein a portion of the gRNAs are complementary to target sequences, such as sequences targeted for depletion. In some embodiments, the method comprises using gRNAs which can base-pair with the targeted sites, wherein the sample is contacted with at least 10² unique gRNAs. In some embodiments the sample is contacted with at least 10², at least 10³, at least 10⁴, at least 10⁵, at least 10⁶, at least 10⁷, at least 10⁸, at least 10⁹, at least 10¹⁰ unique gRNAs. In one exemplary embodiment, the sample is contacted with about 10⁵ unique gRNAs.

In the embodiments provided herein, the methods comprise contacting a sample with at least 10² unique CRISPR/Cas system protein-gRNA complexes, where the unique nature of the complex is determined by the unique nature of the gRNA itself. For example, 2 unique CRISPR/Cas system protein-gRNA complexes may share the same CRISPR/Cas system protein, but the gRNAs differ, even if by only 1 nucleotide. Thus, in some embodiments, the method comprises contacting a sample with at least 10² unique CRISPR/Cas system protein-gRNA complexes. In some embodiments the sample is contacted with at least 10², at least 10³, at least 10⁴, at least 10⁵, at least 10⁶, at least 10⁷, at least 10⁸, at least 10⁹, at least 10¹⁰ unique CRISPR/Cas system protein-gRNA complexes. In one exemplary embodiment, the sample is contacted with about 10³, 10⁴ or 10⁵ unique CRISPR/Cas system protein-gRNA complexes.

In the embodiments provided herein, the methods comprise contacting a sample comprising genomic DNA with a plurality of CRISPR/Cas9 system protein-gRNA complexes, wherein the gRNAs are complementary to sites targeted in the genome for depletion. In some embodiments, the method comprises using gRNAs which can base-pair with the targeted DNA, wherein the target site of interest is spaced at least every 1 bp, at least every 2 bp, at least every 3 bp, at least every 4 bp, at least every 5 bp, at least every 6 bp, at least every 7 bp, at least every 8 bp, at least every 9 bp, at least every 10 bp, at least every 11 bp, at least every 12 bp, at least every 13 bp, at least every 14 bp, at least every 15 bp, at least every 16 bp, at least every 17 bp, at least every 18 bp, at least every 19 bp, 20 bp, at least every 25 bp, at least every 30 bp, at least every 40 bp, at least every 50 bp, at least every 100 bp, at least every 200 bp, at least every 300 bp, at least every 400 bp, at least every 500 bp, at least every 600 bp, at least every 700 bp, at least every 800 bp, at least every 900 bp, at least every 1000 bp, at least every 2500 bp, at least every 5000 bp, at least every 10,000 bp, at least every 15,000 bp, at least every 20,000 bp, at least every 25,000 bp, at least every 50,000 bp, at least every 100,000 bp, at least every 250,000 bp, at least every 500,000 bp, at least every 750,000 bp, or even at least every 1,000,000 bp across a genome of interest.

In the embodiments provided herein, the methods comprise contacting a sample comprising nucleic acids targeted for depletion with a plurality of CRISPR/Cas9 system protein-gRNA complexes, wherein the gRNAs contain a portion that is complementary to the nucleic acids targeted for depletion. In some embodiments the molar ratio of the gRNA:nucleic acids targeted for depletion is 1:1, 5:1, 10:1, 50:1, 100:1, 150:1, 250:1,500:1,750:1,1000:1, 1500:1, 2000:1, 2500:1, 5000:1, 7500:1, or even 10,000:1. In an exemplary embodiment the molar ratio of the gRNA:nucleic acids targeted for depletion is 500:1.

In the embodiments provided herein, the methods comprise contacting a sample comprising nucleic acids targeted for depletion with a plurality of CRISPR/Cas9 system protein-gRNA complexes, wherein the gRNAs are complementary to the nucleic acids targeted for depletion. In some embodiments the weight to weight ratio of the gRNA:nucleic acids targeted for depletion is 1:1, 5:1, 10:1, 20:1, 30:1, 40:1, 50:1, 60:1, 70:1, 80:1, 90:1, 100:1, 150:1, 250:1,500:1,750:1,1000:1, 1500:1, 2000:1, 2500:1, 5000:1, 7500:1, or even 10,000:1. In an exemplary embodiment the weight to weight ratio of the gRNA:nucleic acids targeted for depletion is ranges between 50:1 and 100:1.

### CRISPR/Cas System Proteins

Provided herein are compositions and methods for the depletion of unwanted nucleic acids, and/or enrichment of sequences of interest in a sample. These compositions and methods utilize a nucleic acid-guided nuclease such as a CRISPR/Cas system protein. CRISPR/Cas system proteins include proteins from CRISPR Type I systems, CRISPR Type II systems, and CRISPR Type III systems. CRISPR/Cas system proteins can be from any bacterial or archaeal species. In some embodiments, the CRIPR/Cas system proteins are from, or are derived from CRISPR/Cas system proteins from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

The nucleic acid-guided nuclease proteins can be naturally occurring or engineered versions. Naturally occurring CRISPR/Cas system proteins include Cas9, Cpf1, Cas3, Cas8a-c, Cas10, Cse1, Csy1, Csn2, Cas4, Csm2, and Cm5. In an exemplary embodiment, the CRISPR/Cas system protein comprises Cas9.

A “CRISPR/Cas system protein-gRNA complex” refers to a complex comprising a CRISPR/Cas system protein and a guide RNA. The guide RNA may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a gRNA) that contains crRNA and tracrRNA sequences. A CRISPR/Cas system protein may be at least 60% identical (e.g., at least 70%, at least 80%, or 90% identical, at least 95% identical or at least 98% identical or at least 99% identical) to a wild type CRISPR/Cas system protein. The CRISPR/Cas system protein may have all the functions of a wild type CRISPR/Cas system protein, or only one or some of the functions, including binding activity, nuclease activity, and nicking activity.

The term “CRISPR/Cas system protein-associated guide RNA” refers to a guide RNA as described above (comprising a crRNA molecule and a tracrRNA molecule or comprising a single RNA molecule that includes both crRNA and tracrRNA sequences). The CRISPR/Cas system protein-associated guide RNA may exist as isolated RNA, or as part of a CRISPR/Cas system protein-gRNA complex.

### Cas9

The CRISPR/Cas system protein used in the methods may comprise Cas9. The Cas9 of the present invention can be isolated, recombinantly produced, or synthetic. Cas9 proteins that can be used in the embodiments herein can be found in Ran et al. “in vivo genome editing using Staphylococcus aureus Cas9” Nature. 520, 186-191 (2015), which is incorporated by reference in its entirety.

In some embodiments, the Cas9 is a Type II CRISPR system derived from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

In some embodiments, the Cas9 is a Type II CRISPR system derived from S. pyogenes and the PAM sequence is NGG located on the immediate 3′ end of the target specific guide sequence. The PAM sequences of Type II CRISPR systems from exemplary bacterial species can also include: Streptococcus pyogenes (NGG), Staph aureus (NNGRRT), Neisseria meningitidis (NNNNGA TT), Streptococcus thermophilus (NNAGAA) and Treponema denticola (NAAAAC) which are all usable without deviating from the present invention, see Ran et al., supra.

In one exemplary embodiment, Cas9 sequence can be obtained, for example, from the pX330 plasmid (available from Addgene), re-amplified by PCR then cloned into pET30 (from EMD biosciences) to express in bacteria and purify the recombinant 6His tagged protein.

The “Cas9-gRNA complex” refers to a complex comprising a Cas9 protein and a guide RNA and is one example of a CRISPR/Cas system protein-gRNA complex. A Cas9 protein may be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein may have all the functions of a wild type Cas9 protein, or only one or some of the functions.

The term “Cas9-associated guide RNA” refers to a guide RNA as described above (comprising a crRNA molecule and a tracrRNA molecule or comprising an RNA molecule that includes both crRNA and tracrRNA sequences). The Cas9-associated guide RNA may exist as isolated RNA, or as part of a Cas9-gRNA complex.

### CRISPR/Cas System Protein Nickases

Engineered examples of CRISPR/Cas system proteins also include Cas nickases. A Cas nickase refers to a modified version of a CRISPR/Cas system protein, containing a single inactive catalytic domain. The CRISPR/Cas system protein nickase is Cas9 nickase, Cpf1 nickase, Cas3 nickase, Cas8a-c nickase, Cas10 nickase, Cse1 nickase, Csy1 nickase, Csn2 nickase, Cas4 nickase, Csm2 nickase, or Cm5 nickase. In one embodiment, the CRISPR/Cas system protein nickase is Cas9 nickase.

In some embodiments, a Cas9 nickase can be used to bind to target sequence. The term “Cas9 nickase” refers to a modified version of the Cas9 protein, containing a single inactive catalytic domain, for example, either the RuvC- or the HNH-domain. With only one active nuclease domain, the Cas9 nickase cuts only one strand of the target DNA, creating a single-strand break or “nick”. Depending on which mutant is used, the guide RNA-hybridized strand or the non-hybridized strand may be cleaved. Cas9 nickases bound to 2 gRNAs that target opposite strands can create a double-strand break in the DNA. This “dual nickase” strategy increases the specificity of cutting because it requires that both Cas9/gRNA complexes be specifically bound at a site before a double-strand break is formed.

In an exemplary embodiment, depletion of DNA can be carried out using a Cas9 nickase. In one embodiment, the method comprises: making a DNA sequencing library comprising DNA to be removed (for example human DNA not of interest) and DNA of interest (for example a DNA from an unknown pathogen); designing guide RNAs so that all the DNA to be depleted will have two guide RNA binding sites in close proximity (for example, less than 15 bases apart) on opposite DNA strands; adding Cas9 Nickase and guide RNA to the DNA library. In this embodiment, the Cas9 Nickase recognizes its target sites on the DNA to be removed and cuts only one strand. For DNA to be depleted, two separate Cas9 Nickases can cut both strands of the DNA to be removed (e.g. human DNA) in close proximity; only the DNA to be removed (e.g. human DNA) will have two Cas9 nickase sites in close proximity which creates a double stranded break.

### Dissociable and Thermostable CRISPR/Cas System Proteins

Although CRISPR/Cas System proteins can be used in combination with a library of guide RNAs to efficiently deplete a collection of target DNA, large amounts (e.g. >30 pmoles) of CRISPR/Cas System proteins and guide RNAs may be needed. One reason for this usually >100 fold excess amount over target DNA is that, unlike classical restriction enzymes such as EcoRI, which detach completely from their target DNA after cleavage, CRISPR/Cas System proteins are not completely recycled after completion of the cutting reaction. CRISPR/Cas System proteins, for example Cas9, can remain bound to one of the two daughter DNA product molecules. As a result, more CRISPR/Cas System proteins and gRNA may need to be provided in order to achieve complete depletion of unwanted DNA.

In some embodiments, to overcome this problem, dissociable CRISPR/Cas System proteins are provided herein. For example, upon cleavage of targeted sequences, the CRISPR/Cas System protein can be made to dissociate from the gRNA, or from the target. In some embodiments, the dissociation is induced by elevating the temperature of the reaction. This can act to increase processivity of the enzyme, by allowing it to complex with available gRNAs, re-associate with additional target sequences and generate additional cleaved target sequences.

In some embodiments to overcome this problem, thermostable CRISPR/Cas System proteins are provided herein. In such embodiments, the reaction temperature is elevated, inducing dissociation of the protein; the reaction temperature is lowered, allowing for the generation of additional cleaved target sequences. In some embodiments, thermostable CRISPR/Cas system proteins maintain at least 50% activity, at least 55% activity, at least 60% activity, at least 65% activity, at least 70% activity, at least 75% activity, at least 80% activity, at least 85% activity, at least 90% activity, at least 95% activity, at least 96% activity, at least 97% activity, at least 98% activity, at least 99% activity, or 100% activity, when maintained for at least 75° C. for at least 1 minute. In some embodiments, thermostable CRISPR/Cas system proteins maintain at least 50% activity, when maintained for at least 1 minute at least at 75° C., at least at 80° C., at least at 85° C., at least at 90° C., at least at 91° C., at least at 92° C., at least at 93° C., at least at 94° C., at least at 95° C., 96° C., at least at 97° C., at least at 98° C., at least at 99° C., or at least at 100° C. In some embodiments, thermostable CRISPR/Cas system proteins maintain at least 50% activity, when maintained at least at 75° C. for at least 1 minute, 2 minutes, 3 minutes, 4 minutes, or 5 minutes. In some embodiments, a thermostable CRISPR/Cas system protein maintains at least 50% activity when the temperature is elevated, lowered to 25° C.-50° C. In some embodiments, the temperature is lowered to 25° C., to 30° C., to 35° C., to 40° C., to 45° C., or to 50° C. In one exemplary embodiment, a thermostable enzyme retains at least 90% activity after 1 min at 95° C.

In some embodiments, the thermostable CRISPR/Cas system protein is thermostable Cas9, thermostable Cpf1, thermostable Cas3, thermostable Cas8a-c, thermostable Cas10, thermostable Cse1, thermostable Csy1, thermostable Csn2, thermostable Cas4, thermostable Csm2, or thermostable Cm5. In some embodiments the thermostable CRISPR/Cas system protein is thermostable Cas9.

In one exemplary embodiment, thermostable Cas9 complexed with a guide RNAs (for example a gRNA library against human DNA) can be applied to a sequencing library of DNA mixture (containing for example 95% human DNA and 5% viral DNA). After allowing Cas9 to digest for a period of time, the temperature of the sample mixture can be elevated, for example up to 95° C. or greater, which can cause DNA denaturation, as well as dissociation of gRNA and Cas9 from the DNA targets. The binding of Cas9 to gRNAs can be increased so that the Cas9-gRNA dissociates from the DNA target as an intact complex, despite DNA denaturation. Dimethyl sulfoxide can be added to reduce the temperature required for DNA denaturation, so that the Cas9 protein structure is not affected. Cas9 preferentially binds to target sites that have not been cut, and a thermostable Cas9 can retain activity after boiling. Because of these features, by elevating the temperature, for example to 100° C., and cooling down the reaction to, for example, 37° C., a thermostable Cas9 can remain capable of binding to its gRNA and cutting more of its substrate. By allowing the recycling of Cas9, the depletion efficiency can be increased, and as less Cas9 will be needed in the reaction, the off-target (non-specific) cleavage probability can also be decreased.

Thermostable CRISPR/Cas System proteins can be isolated, for example, identified by sequence homology in the genome of thermophilic bacteria Streptococcus thermophilus and Pyrococcus furiosus. CRISPR/Cas system genes can then be cloned into an expression vector. In one exemplary embodiment, a thermostable Cas9 protein is isolated.

In another embodiment, a thermostable CRISPR/Cas system protein can be obtained by in vitro evolution of a non-thermostable CRISPR/Cas system protein. The sequence of a CRISPR/Cas system protein can be mutagenized to improve its thermostability. In some embodiments, this can be achieved by site-directed mutagenesis to remove excess loop sequences, increasing the number of ionic bridges between protein domains, or by diluting into droplets and PCR to create a pool of potential mutants. In one exemplary embodiment, a thermostable Cas9 is produced by in vitro evolution of a non-thermostable Cas9.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

### Example 1: Pre-Depletion of Human DNA

The analysis of nucleic acid sequences in samples comprising complex mixtures of nucleic acids, e.g., clinical samples, forensic samples, metagenomic samples, can be challenging. Particularly challenging is the identification of rare sequences of interest. For example, detecting nucleic acids from pathogens in a sample from a host either requires significant depth of sequencing, which may be cost prohibitive, or methods to partition or separate the sequences of interest from other sequences. Therefore, the inventors hypothesized that pre-depleting samples with a high proportion of known sequences could lead to increased enrichment of sequences of interest for use in downstream analyses, e.g., next generation sequencing.

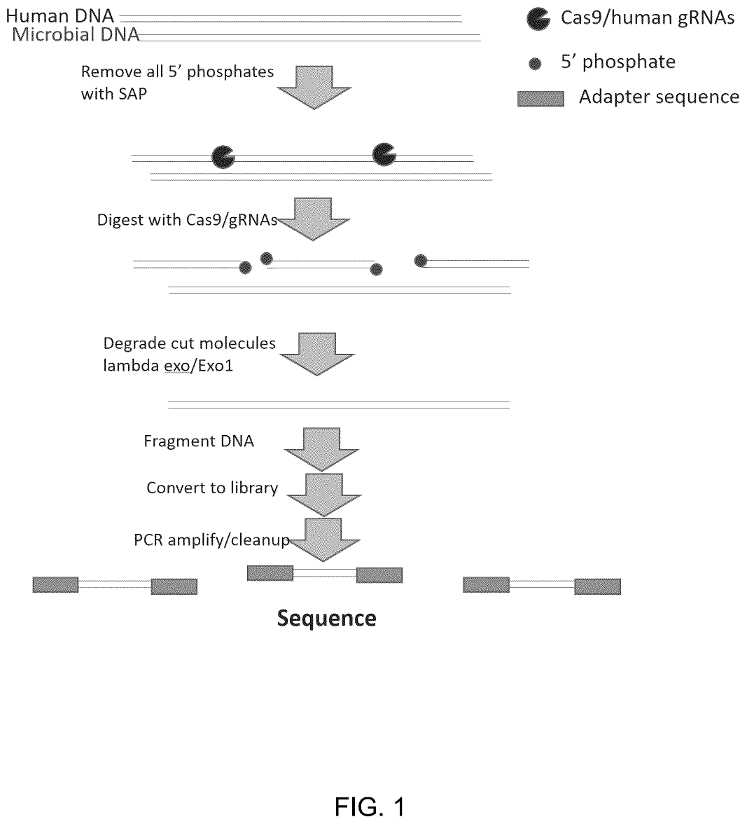

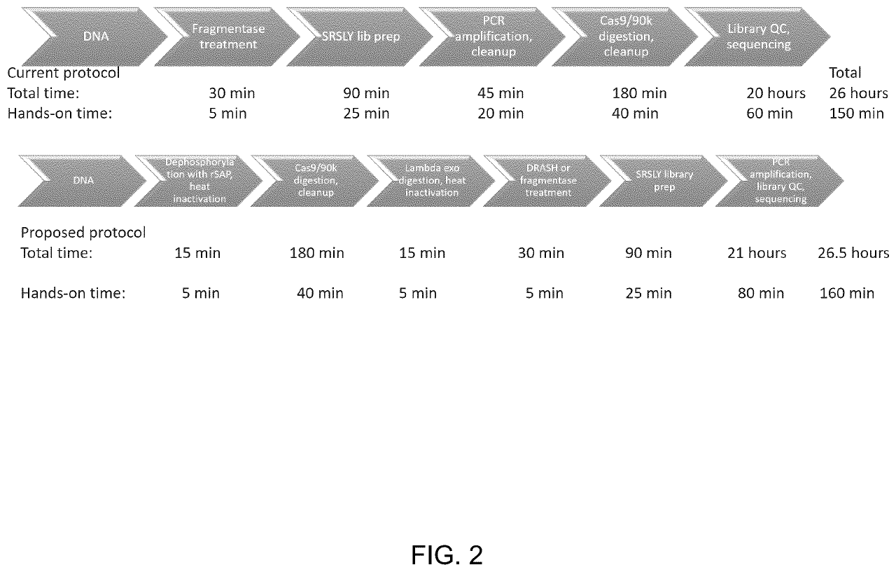

The inventors designed methods to deplete human DNA in a targeted manner. FIG. 1 shows one exemplary embodiment of the protocol. Starting with a sample of mixed human and microbial DNA the nucleic acids in the sample are dephosphorylated with a phosphatase, e.g., shrimp alkaline phosphatase (SAP), to generate DNA sequences without 5′ phosphates. Next, the dephosphorylated DNA in the sample is contacted with a plurality of CRISPR/Cas9 gRNA complexes. The gRNAs are suitably designed such that the target-specific portion of the gRNAs are complementary to human (host) DNA. Therefore, the CRISPR/Cas9-gRNA complexes create double-stranded breaks at a variety of loci in the human DNA, thereby creating an increased number of host DNA molecules with 5′ phosphates. Next, the sample is contacted with lambda exonuclease which degrades DNA with 5′ phosphates, resulting in reduced numbers of human host DNA molecules in the sample. The sequences of interest may then be cleaned up by, for example, using a PCR cleanup kit and ligated to adapters for adapter-mediated PCR. Following adapter-mediated PCR, the sample is enriched for the sequences of interest. Alternatively, after cleanup, the sample may be further enriched for sequences of interest, or further depleted of host DNA.

### Example 2: Sequence Pre-Depletion in Combination with Further Downstream Depletion

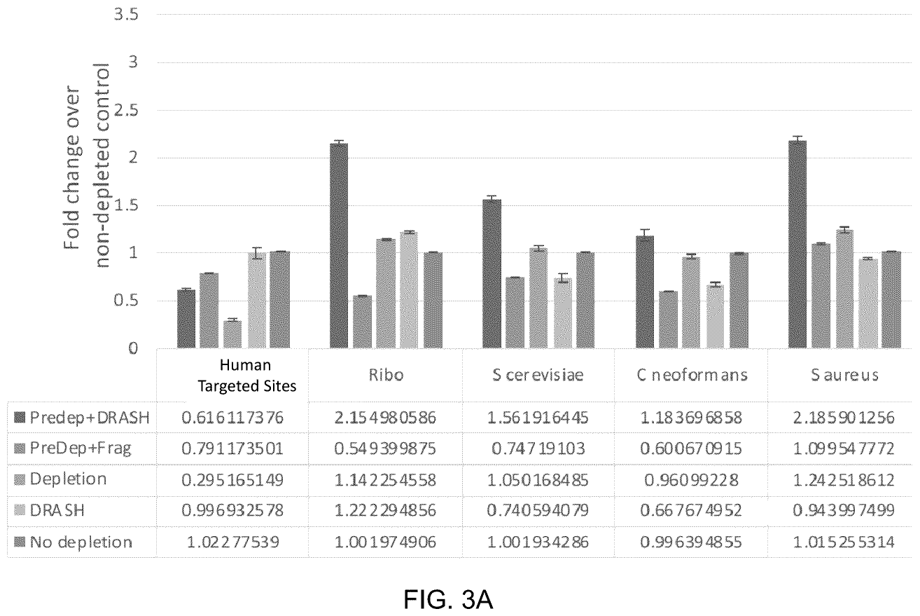

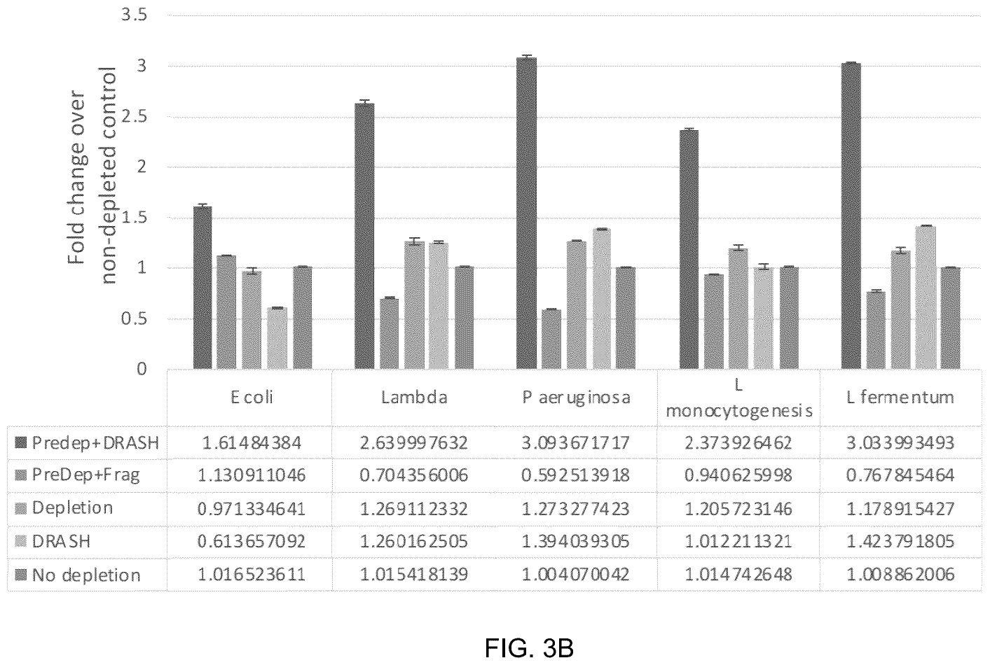

The inventors hypothesized that the pre-depletion strategy demonstrated in FIG. 1 and Example 1 would synergize with other novel sequence enrichment and depletion strategies. Therefore, the inventors developed a microbial spike-in system to test the pre-depletion strategy of Example 1 compared to alternative strategies, as well as in combination with other strategies. The inventors used a synthetic plasma matrix control (Qnostics) as a base solution to spike in 99% human DNA (Promega), 0.5% lambda phage DNA, and 0.5% microbiomic mix (Zymo) comprising Saccharomyces cerevisiae, Cryptococcus neoformans, Staphylococcus aureus, Escherichia coli, Pseudomonas aeruginosa, Listeria monocytogenes, and Lactobacillus fermentum (FIG. 3A and FIG. 3B). Thus, the inventors developed a surrogate for human plasma that largely comprises human DNA, but also comprises DNA from commensal or opportunistic organisms, e.g., Escherichia coli, Lactobacillus fermentum, and pathogenic organisms, e.g., Cryptococcus neoformans, Staphylococcus aureus, Pseudomonas aeruginosa, and Listeria monocytogenes.

The inventors either performed no depletion (control), the pre-depletion described in FIG. 1 and example 1 alone followed by fragmentation, DRASH alone, pre-depletion followed by DRASH, or standard depletion.

DRASH is a depletion strategy previously developed by the inventors that relies on differential CpG methylation between mammalian, e.g., human, DNA and microbial DNA. Vertebrate DNA comprises many more CpG methylation sites than microbial DNA. Therefore, the inventors previously developed a strategy to enrich microbial DNA by contacting a sample with one or more DRASH enzymes, i.e., enzymes selected from the group consisting of AluI, DdeI, HpyCH4IV, RsaI, and Sau3AI, which are CpG sensitive and will not cut DNA at CpG sites. Thus, the microbial DNA is cut significantly more by the DRASH enzymes and adapters are ligated to the cut microbial DNA and may be enriched by adaptor-mediated PCR.

Standard depletion refers to depletion of host sequences by cutting with a plurality of CRISPR/Cas9 gRNA complexes targeted to the host genome described in U.S. Pat. No. 10,774,365, which is incorporated herein by reference in its entirety.

Surprisingly, the inventors discovered that the novel pre-depletion method of the instant disclosure synergized with the DRASH depletion method to increase the number of sequence reads from the S. cerevisiae and E. coli greater than 1.5 fold, reads from S. aureus and L. monocytogenes greater than 2 fold, reads from lambda phage greater than 2.5 fold, and reads from P. aeruginosa and L. fermentum about 3 fold over samples without any depletion (FIG. 3A and FIG. 3B). At the same time, the sites targeted for depletion in human (“Human Targeted Sites”) and ribosomal (“Ribo”) nucleic acids decreased their prevalence.

Increased E. coli signal was detected and was caused by presence of extra E. coli from the Cas9 prep, confirmed by mapping to pET30Cas9 plasmid. Subtracting these reads from E. coli mapping mitigated the extra E. coli signal.

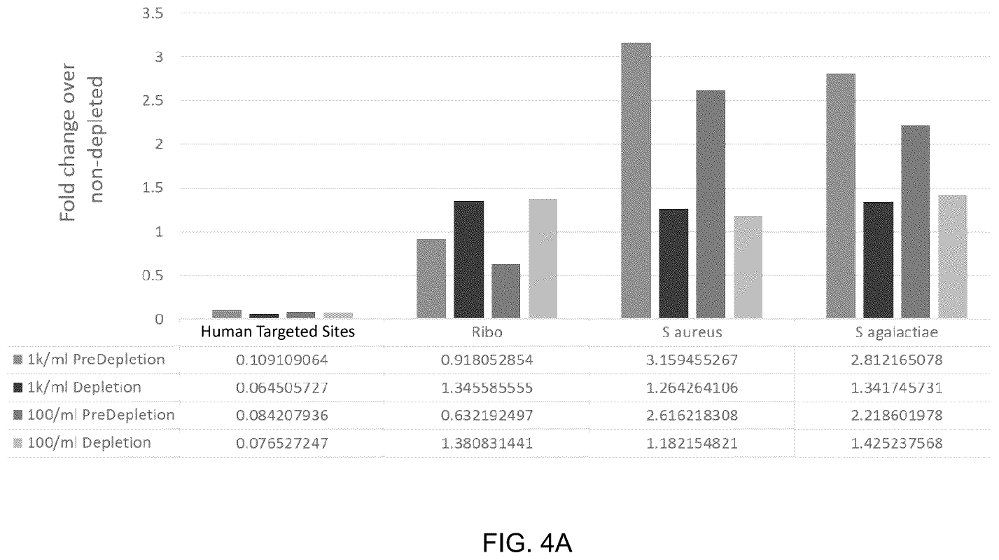

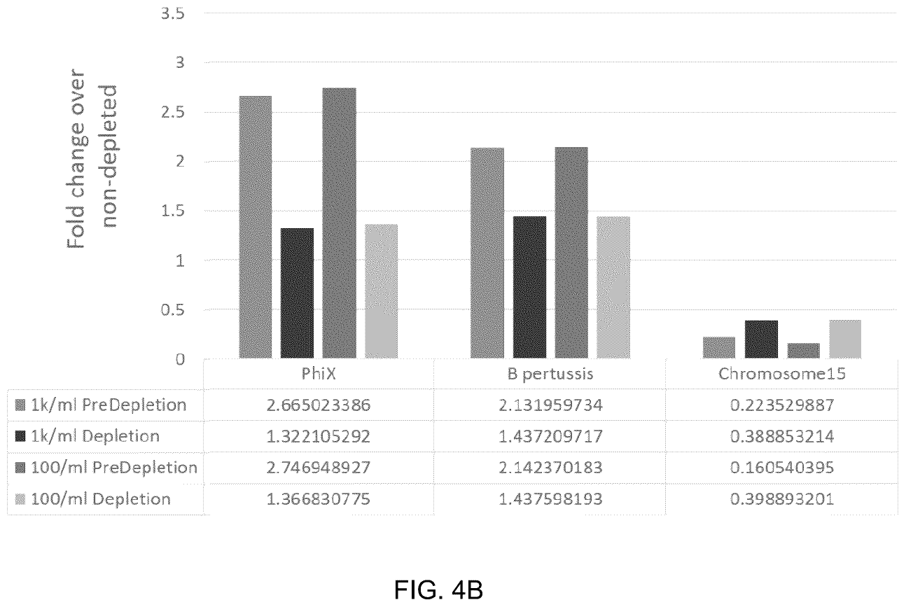

Next, the inventors tested the novel pre-depletion methods of the instant disclosure in comparison with the standard depletion strategy described above as a method to detect rare microbial DNA in a simulated plasma matrix. The sample was prepared similarly to that described above, and included Staphylococcus aureus, Streptococcus agalactiae, Phi X phage (a single-stranded DNA phage), and Bordetella pertussis, at concentrations of either 1000 organisms per ml or 100 organisms per ml and performed either the novel pre-depletion method disclosed herein, or the standard depletion discussed above. Surprisingly, the inventors discovered that the novel pre-depletion method increased the recovery of reads mapped to the microorganisms by at least 2-fold for each of the organisms at both the 1000/ml and 100/ml concentrations, as compared to non-depleted control samples (FIG. 4A and FIG. 4B). In addition, standard depletion and pre-library depletion performed similarly at the targeted sites, while pre-library depletion performed better than standard depletion across human genome more broadly, as shown in the results for Chromosome 15.

### Example 3: Depletion and Protection of Single-Stranded DNA in Combination with Pre-Depletion

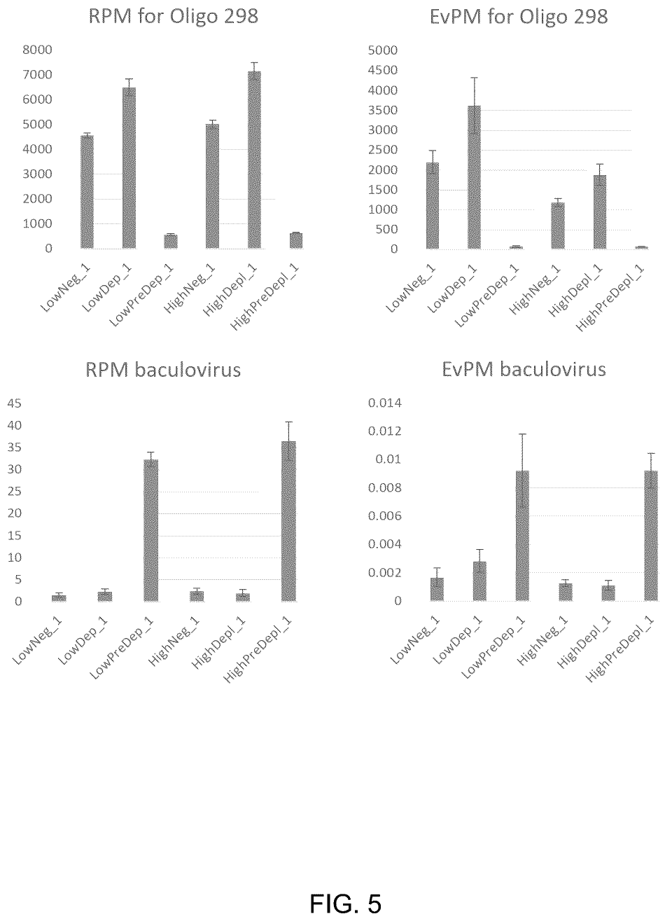

To test the level of depletion of single-stranded DNA in a sample, the inventors spiked in single-stranded oligonucleotides (“Oligo 298”), and a large genome virus, baculovirus, into the simulated plasma matrix described above and performed either no depletion, standard depletion, or the novel pre-depletion method and sequenced the resulting DNA library. The pre-depletion method significantly reduced the reads per million (RPM) and genome equivalents per million (EvPM) that mapped to the single-stranded oligo 298 as compared to both no depletion and the standard depletion. See FIG. 5. Interestingly, the pre-depletion significantly enriched the reads mapping to baculovirus as compared to either no depletion or standard depletion. See FIG. 5. The inventors attribute this result to the fragmentation of DNA post-enrichment, which artificially increases the number of reads mapping to the baculovirus genome which, with reference to the oligonucleotides, is comparatively large.

The inventors hypothesized that the disclosed pre-depletion method could be modified to prevent degradation of single-stranded DNA, which is naturally present in relevant sources, e.g., from viruses with single-stranded genomes. Therefore, the inventors incorporated a second-strand synthesis step into the novel pre-depletion method. The inventors prepared a mixture of lambda phage DNA (double-stranded) and 200 base pair long DNA oligonucleotides (single-stranded), at a 1:1 ratio. One aliquot of the mixture was contacted with random hexamers and Klenow DNA polymerase to perform second-strand synthesis for any single-stranded DNA present. Then, each of the samples were dephosphorylated with SAP, cut with the restriction enzyme EcoRV, and used directly, or further contacted with lambda exonuclease, which degrades double-stranded DNA with 5′ phosphates, contacted with exonuclease 1, which degrades single-stranded DNA, or a combination. The inventors demonstrated that second-strand synthesis offered protection for single-stranded DNA in the sample. See FIG. 6.

What is claimed is:

1. A method of enriching a sample for sequences of interest, comprising:
a) providing a sample comprising sequences of interest and targeted sequences for depletion;
b) contacting the sample with a phosphatase to remove 5′ phosphates on the sequences of interest and the targeted sequences for depletion to generate a sample comprising sequences lacking 5′ phosphates;
c) contacting the sample comprising sequences lacking 5′ phosphates of step (b) with a plurality of nucleic acid-guided nuclease-guide nucleic acid (gNA) complexes, wherein the guide nucleic acids (gNAs) include a region complementary to targeted sequences for depletion, and whereby the targeted sequences for depletion are cleaved to generate a sample comprising sequences of interest and cleaved targeted sequences for depletion; and
d) contacting the sample of step (c) with at least one exonuclease capable of targeting 5′ phosphate containing sequences for degradation and degrading the cleaved targeted sequences for depletion to generate a sample enriched for the sequences of interest.

2. The method of claim 1, further comprising:
e) fragmenting the nucleic acids in the sample enriched for the sequences of interest from step (d) to generate a fragmented sample.

3. The method of claim 2, wherein the fragmenting step is completed by contacting the sample of step (d) with a fragmentase to generate the fragmented sample.

4. The method of claim 2, wherein the fragmenting step is completed by contacting the sample of step (d) with one or more CpG methylation-sensitive restriction enzymes to generate cut sites or nick sites in the DNA in the sample at enzyme recognition sites that lack CpG methylation to generate the fragmented sample.

5. The method of claim 4, wherein the one or more CpG methylation-sensitive restriction enzyme comprises a restriction enzyme selected from the group consisting of AatII, AccII, AluI, Aor13HI, Aor51HI, BspT104I, BssHII, Cfr10I, ClaI, CpoI, DdeI, Eco52I, HaeII, HapII, HhaI, HpyCH4IV, MluI, NaeI, NotI, NruI, NsbI, Nt.CviPII, PmaCI, Psp1406I, PvuI, RsaI, SacII, SalI, SmaI, SnaBI, and Sau3AI.

6. The method of claim 4, wherein the one or more CpG methylation-sensitive restriction enzyme comprises at least one of AluI, DdeI, HpyCH4IV, RsaI, and Sau3AI.

7. The method of claim 4, wherein the one or more CpG methylation-sensitive restriction enzyme comprises all five of AluI, DdeI, HpyCH4IV, RsaI, and Sau3AI.

8. The method of any one of claims 2-7, wherein the fragmented sample is subjected to serial depletion by repeating steps (b) through (d) of claim 1 using the fragmented sample.

9. The method of any one of claims 2-8, further comprising ligating adapters to the sequences in the fragmented sample to generate adapter-ligated sequences.

10. The method of claim 9, further comprising amplifying the adapter-ligated sequences using adapter-specific PCR, thereby further enriching for the sequences of interest.

11. The method of any one of claims 1-10, further comprising generating a library comprising adapter-ligated sequences from the sample enriched for the sequences of interest.

12. The method of any one of claims 9-11, further comprising sequencing the adapter-ligated sequences.

13. The method of any one of claims 1-12, further comprising performing second strand synthesis on the sample of step (a) prior to step (b).

14. The method of claim 13, wherein the second strand synthesis is performed by contacting the sample of step (a) with random hexamers and Klenow DNA polymerase.

15. The method of claim 14, wherein after step (b) the sample is further contacted with a blunt-end generating restriction enzyme.

16. The method of claim 15, wherein the blunt-end generating restriction enzyme is EcoRV.

17. The method of any one of claims 1-16, further comprising treating the sample enriched in sequences of interest with an exonuclease capable of degrading single stranded DNA.

18. The method of claim 17, wherein the exonuclease is ExoI.

19. The method of any one of claims 1-18, wherein the nucleic acid-guided nuclease-gNA complexes are Crispr/Cas system protein-gRNA complexes.

20. The method of any one of claims 1-19, wherein the phosphatase is shrimp alkaline phosphatase (SAP) or recombinant SAP (rSAP).

21. The method of any one of claims 1-20, wherein the exonuclease in step (d) is lambda exonuclease.

22. The method of any one of claims 1-21, further comprising after step (d) contacting the sample with an exonuclease specific for single-stranded DNA, wherein the exonuclease is exo I.

23. The method of any one of claims 1-22, wherein the sequences of interest comprise less than 50% of the sample.

24. The method of any one of claims 1-23, wherein the sequences of interest and targeted sequences for depletion are DNA.

25. The method of any one of claims 1-24, wherein the sample is selected from the group consisting of a human sample, clinical sample, a forensic sample, an environmental sample, a metagenomic sample, and a food sample.

26. The method of any one of claims 1-25, wherein the sample comprises host nucleic acid sequences targeted for depletion and non-host nucleic acid sequences of interest.

27. The method of claim 26, wherein the non-host nucleic acid sequences comprise microbial nucleic acid sequences.

28. The method of claim 27, wherein the microbial nucleic acids sequences are derived from bacteria, archaea, fungi, or a eukaryotic parasite.

29. The method of any one of claims 1-28, wherein the gNAs include a region complementary to DNA corresponding to ribosomal RNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

30. The method of any one of claims 1-29, wherein the sample is selected from whole blood, plasma, serum, tears, saliva, mucous, cerebrospinal fluid, teeth, bone, fingernails, feces, urine, tissue, and a biopsy.

31. The method of any one of claims 1-30, further comprising generating a double-stranded or single-stranded DNA library comprising the enriched sequences of interest.

32. The method of claim 31, further comprising amplifying, sequencing, or cloning the DNA library.

33. A kit comprising:
a) a phosphatase;
b) a plurality of gNAs comprising a region complementary to targeted sequences for depletion; and
c) an exonuclease.

34. The kit of claim 33, further comprising:
d) One or more CpG methylation-sensitive restriction enzyme selected from the group consisting of AatII, AccII, AluI, Aor13HI, Aor51HI, BspT104I, BssHII, Cfr10I, ClaI, CpoI, DdeI, Eco52I, HaeII, HapII, HhaI, HpyCH4IV, MluI, NaeI, NotI, NruI, NsbI, Nt.CviPII, PmaCI, Psp1406I, PvuI, RsaI, SacII, SalI, SmaI, SnaBI, and Sau3AI.

35. The kit of claim 34, wherein the one or more CpG methylation-sensitive restriction enzyme comprises at least one of AluI, DdeI, HpyCH4IV, RsaI, and Sau3AI.

36. The kit of claim 35, wherein the one or more CpG methylation-sensitive restriction enzyme comprises all five of AluI, DdeI, HpyCH4IV, RsaI, and Sau3AI.

37. The kit of any one of claims 33-36, wherein the phosphatase is shrimp alkaline phosphatase (SAP) or recombinant SAP (rSAP).

38. The kit of any one of claims 33-37, wherein the exonuclease is lambda exonuclease, exonuclease 1 (EXO1) or both.

39. The kit of any one of claims 33-38, wherein the gNAs comprise gRNAs.

40. The kit of any one of claims 33-39, wherein the targeted sequences for depletion comprise human DNA sequences.

41. The kit of claim 40, wherein the targeted sequences for depletion comprise ribosomal RNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

42. The kit of any one of claims 33-41, further comprising DNA library preparation reagents.
